(19) United States
(12) Patent Application Publication
(10) Pub. No.: US 2004/0115766 A1
(43) Pub. Date: Jun. 17, 2004

(54) Method for producing proteins by fermentation of microorganisms from the thermus family the protein mixture thus obtained and cosmetic compositions containing same

(76) Inventor: Karl Lintner, Paris (FR)

Correspondence Address: YOUNG & THOMPSON, 745 SOUTH 23RD STREET 2ND FLOOR, ARLINGTON, VA, 22202

(21) Appl. No.: 10/468,081

(22) PCT Filed: Feb. 7, 2002
(86) PCT No.: PCT/FR02/00488

(30) Foreign Application Priority Data
Feb. 21, 2001 (FR) 01/02442

(51) Int. Cl. C12P 21/06; C12N 1/20

(52) U.S. Cl. 435/69.1; 435/252.3

(57) ABSTRACT

The use of fermentation media for microorganisms from the Thermus family, in particular microorganisms collected in marine waters close to very deep hydrothermal sources, especially those obtained with the GY1211 strain. Cosmetic compositions containing one or more types of fermentation media for the microorganisms and which are used, in particular, for skin care, notably for the face, body and scalp, and hair care and in order to modulate the cutaneous concentration of ceramides, to stimulate the immune system, to provide protection by means of the chaperone effect and a detoxifying and anti-free radical effect, particularly in relation to the oxygen peroxide.

The skin is the-first image that each of us offers to the view of others, and at all times its appearance has been a subject of preoccupation.

In the course of life, both professional and private, exposure to the sun as well as various pollutants gives rise to chemical and physical attacks which lead to the production among a list of factors of different forms of oxygen radicals. These latter, particularly at the cutaneous level, give rise to irreversible damage whose consequences are at least erythemas, sunstroke, prematuring aging of the skin and, at the most, skin cancer.

The aggravation of acne manifestations, the loss of cutaneous flexibility, drying of the skin and the appearance of wrinkles correspond to the first macroscopic manifestations of aging. At the level of the cutaneous cells, the molecular modifications that appear upon first exposure to the sun, that can be countered only by invasive and very sophisticated methods, are thus all the more pernicious because they are not detectable by the general population.

The sun is not the only thing responsible for the production of free radicals of oxygen. A large number of vital biochemical reactions (for example oxidation of amino acids or of fatty acids, the synthesis of prostoglandins . . . ), give rise quite naturally to these hyper-reactive forms of oxygen, which are extremely toxic for the tissues in general and for the skin in particular.

Human beings are naturally protected against the deleterious effects of oxygen radicals, by physiological regulations, developed in the course of evolution: progressive tanning starting in the spring, against the sub-erythematic cutaneous consequences, the presence of anti-radical enzymes such as superoxide-dismutases and catalases, decrease in the inflammatory reaction and/or immune reaction, thickening of the epidermis . . . .

To solve these problems, the cosmetic industry has placed on the market all sorts of products containing either solar filters, compositions including natural enzymes present in the skin such as SOD (superoxide-dismutase), vegetable extracts and others.

Another natural enzyme, catalase, cannot be used in cosmetic and dermopharmaceutical compositions, because it appears (under No. 74) in the list of substances which cannot enter into the composition of cosmetic products of annex II(1) of European Directive 93/35 of Jun. 14, 1993.

It is thus logical in cosmetic and dermo-pharmaceutical industries to supply products which help the skin better to withstand, even to correct and to repair, the deleterious effects of this chemical pollution.

The invention which is the object of the present application, resides in the discovery that it is possible to obtain, by biotechnological techniques, active principles having powerful anti-radical effects from microorganisms conventionally used.

The microorganisms in question are part of new strains of thermophilic entities discovered in submarine thermal vents.

These bacteria are capable of surviving at temperatures at which normally the proteins are denatured as well as under pressures such as the cells could not maintain homeostasis between their internal and external media.

Until now, thermophilic microorganisms are used in biotechnology to prepare DNA usable in new amplification techniques called “PCR” (Polymerase Chain Reaction) from lysates of cells.

Thus, the production of active principles from such microorganisms is of interest in two principal ways:

Phylogenetic studies comparing the RNA sequences of ribosomals 16S have shown that all the procaryots no doubt derive from a same common thermophilic ancestor. These thermophilic entities are thus probably constituted of original substances and substances of origin adapted to have their new therapeutic or even cosmetic effects.

Certain metabolites produced by these thermophilic bacteria are thermostable, which is to say that their properties persist at high temperature. The lifetime of the activity of the molecules could cause them to be stable over the long term in the final cosmetic product.

The process for production of proteins by fermentation of microorganisms according to the present application uses a strain of the genus Thermus, in particular Thermus thermophilus, species GY1211, and have isolated it from a submarine hydrothermal vent (at 2000 meters depth) in the east Pacific ridge in the Guayanas basin (Gulf of California). These are gram negative bacilli, whose size is substantially equal to 0.5 μm×10 μm and which are aerobic, heterotrophic, non-sporulated, thermophilic, barotolerant and which, when cultured, give colonies that are orange, round, regular, smooth, not scalloped.

The strain is registered under the number I-2715 at the National Collection of Micro-organism Cultures of the Pasteur Institute of Paris.

We have discovered that these strains of thermophilic entities are capable of producing or supplying molecules which have numerous physiological activities usable in the cosmetic and dermopharmaceutical fields.

It is particularly interesting to cultivate them either so that they excrete these molecules or to use the obtained biomass.

The following examples indicate the results which we have obtained to optimize the culture conditions to achieve these two objectives.

Optimization of the Production of Biomass

For an industrial application it was necessary to obtain the maximum biomass whilst having the most simple and economical culture medium possible. It was necessary to evaluate the influence of the different physico-chemical parameters on the bacterial culture and thereby to determine the limiting factors which are listed below:

Composition of nutritional elements in the GY/01 medium: yeast extract and NH₄Cl. To avoid conventional problems such as coloring and odor, it is necessary to limit their concentration to the minimum necessary. As to yeast extract, it has been determined that the concentration of 0.25% is the limiting concentration and that at 1.0%, there is obtained no more biomass than at 0.5%. The concentration of yeast extract selected is thus 0.5%.

The optimum concentration of use of NH₄Cl that is selected is 0.3%.

Composition in salts and oligo-elements Thermus being a marine bacterium, the content of sodium chloride is very important and, under our conditions, we have chosen to use 1.0% of NaCl in the culture medium, higher concentrations reducing the quantity of biomass obtained.

Moreover, we have chosen to carry out fermentation between 72 and 75° C., with agitation, with a pH equal to 7.2 without adjustment during culturing.

Under these conditions, the time of doubling the population is equal to 47 minutes with a quantity of growth μₘ equal to 0.386 (H⁻¹).

Optimization of the Production of Proteins

Most proteins are generally endocellular.

After filtration (0.2 μm), a unidimensional electrophoresis of the fermented medium with GY1211 demonstrates the sole presence of several protein bands, only if the medium has first been concentrated 20 times. It was thus necessary to find a technical way to cause the proteins to leave the cell.

Numerous experiments have permitted us to develop several procedures of protein extraction in quantity, that are satisfactory and altogether compatible with industrial use.

One procedure giving entire satisfaction is the following: after culturing, there is added Crodasinic LS30 (CRODA) at the final concentration of 0.1% for 30 minutes.

The dosage of the proteins in the culture medium, by simple calorimetry, indicates a minimum concentration of proteins of Thermus equal to 0.2% when unidimensional electrophoresis demonstrates the presence of numerous bands and hence the large variety of proteins extracted. It is to be noted that this treatment does not alter the quaternary structure of the proteins because the enzymatic activities measured are not altered. Finally, it is important to note that complementary tests have demonstrated the invariability, both qualitative and quantitative, of the electrophoretic profile of the proteins obtained after a thermal treatment of 20 minutes at 100° C., which quality demonstrates the pertinence of the fermentation protocol used because the proteins obtained have the same characteristics as those of the original Thermus microorganism and particularly those of the strain GY1211.

Alternatively to this detersive treatment with Crodasinic LS30, can be employed the conventional technique by high pressure homogenization, even though this procedure is longer.

Thus, we have succeeded in optimizing the production of biomass and the quality of the proteins sought from Thermus, thanks to a fermentation of which all the parameters have been optimized by the use of Crodasinic LS30 at the end of fermentation as a simple and less costly way to recover the bacterial proteins in the medium.

Although a single example of retrieval has been described in this patent application, other processes of fermentation and extraction of proteins can be used and thus any other process for fermentation and extraction of proteins can be used within the scope of this application.

For example, to increase further the quantity of proteins in the product, it is possible to concentrate the fermentation medium by any physical or chemical process such as for example, without it being limiting, a 0.2 μm filtration to concentrate the bacteria before the addition of Crodasinic LS30, or else by filtration at the threshold of cutting of small molecular weight (tangential ultrafiltration at the threshold 20 kD), which permits eliminating salts (potential cause of precipitation), small peptides (sometimes causing coloration and undesirable odors) and a portion of the Crodasinic LS30. Those skilled in the art will be able to adapt and modify the techniques to optimize their use according to specific needs of production.

The product obtained after fermentation and filtration can undergo various types of treatment: clarification, concentration or dilution, preservation, purification, fractionation by precipitation, by chromatography, lyophilization or spraying.

One of the preferred embodiments of the invention is constituted by the fermented medium of GY1211 after detersive treatment and concentration by ultrafiltration.

The products obtained after one or the other or several of the treatments (solutions, powders, crystals, emulsions, . . . ) can be used in any conventional galenic form in cosmetology and dermopharmacy. The two following compositions are given by way of non-limiting example.

### EXAMPLE NO. 1

Day Cream

| | |
|---|---|
| Volpo S20 | 2.4 |
| Volpo S2 | 2.6 |
| Prostearyl 15 | 8.0 |
| Beeswax | 0.5 |
| Abil ^{®} ZP2434 | 3.0 |
| Propylene glycol | 3.0 |
| Carbopol ^{®} 941 | 0.25 |
| Triethanolamine | 0.25 |
| Fermented medium | 5.0 |
| Water and preservatives QSP | 100 g |

### EXAMPLE NO. 2

Body Milk

| | |
|---|---|
| Crillet 3 | 2.5 |
| Novol | 0.9 |
| Fluilan | 2.5 |
| Carbopol 940 | 0.3 |
| Beeswax | 2.0 |
| Triethanolamine | 0.1 |
| Glycerin | 5.0 |
| Fermented medium | 3.0 |
| Water and preservatives QSP | 100 g |

Among numerous beneficial effects discovered during the use of this product, the more important deserving to be mentioned are the regulation of the concentrations of cutaneous ceramides, an immuno-stimulative effect of the CD 40 observed in vitro on human keratinocytes, a protective effect of the chaperon protein type, and a detoxifying effect as to different hyper-reactive free radicals by the presence of menaquinone in the culture medium according to this application, of which the following example demonstrates the effect as to only hydrogen peroxide but which is not the only radical in question.

### EXAMPLE NO. 3

“Catalase-Like” Activity in Vitro

The simplest method to demonstrate the “catalase-like” activity consists in following by spectrophotometry the degradation of H₂O₂ according to the following equation:

$${H}_{2} {O}_{2} {⟶}^{Catalase} {H}_{2} O + \frac{1}{2} {O}_{2}$$

The H₂O₂ absorbs at 240 nm, hence the drop in DO at 240 nm is proportional to the degradation of H₂O₂, which permits, by comparison with a scaled spectrum from bovine catalase, measuring the enzymatic or pseudoenzymatic activity of the unknown specimen.

The following table assembles the pseudoenzymatic activities of the catalase type (means±SEM) of the culture media treated either by Crodasinic or by high pressure (APV material), during 8 independent experiments carried out at 25° C. in which the [C] Factor indicates the concentration of the produced medium.

Activity expressed in U/ml

| Specimen: | [C] Factor | Crodasinic | APV |
|---|---|---|---|
| Unfermented medium | ×1 | 0 | 0 |
| Fermented medium | ×1 | 0.37 | 0.45 |
| Ultrafiltered | ×10 | 4.91 | 5.02 |
| fermented medium | | | |

There is observed for all the specimens fermented with Thermus an enzymatic activity whilst the unfermented culture media themselves are without this. Moreover, the ratio between the measured activities is coherent with that of the produced concentration.

It is also possible to note that the treatments used are equivalent in terms of enzymatic activity obtained.

Finally, it is particularly important to point out that if the activity of the bovine catalase falls with the temperature at which the test is carried out (−25% of activity at 45° C. versus 25° C.), the pseudoenzymatic activity of the fermentation media with Thermus increases considerably with temperature whilst at 45° C. it is multiplied by a factor of 4.5. Because in its natural environment, Thermus GY1211 survives at temperatures of the order of 70° C., it is logical that these defense mechanisms be particularly efficacious at high temperature. From a cosmetic point of view, this “activation” by heat constitutes a considerable advantage. Thus, it is principally in summer that one is exposed to the sun and one has the most need for a catalase-like activity. However, under these conditions, the temperature of the skin can reach temperatures higher than 30° C.

Activity has not been measured beyond 45° C., because we would then be talking about conditions incompatible with the temperature of the skin in vivo.

Thus, the fermented medium described above which has enzymatic activities that are peroxydasic, catalase-like, antiradical, inhibitive of glycation, and that modifies the synthesis of collagen, can be used for care of the skin, particularly to guard against the deleterious effects of free radicals and against any of their consequences for aging which is cutaneous, physiologic or premature, during exposure to natural UV or else for the care of the skin, in particular the face, the body, the scalp and the hair; to modulate the cutaneous concentration of ceramides, to stimulate the immune system, to obtain a protective effect by the chaperone effect and as a detoxifier for the different hyper-reactive free radicals and in particular ozone, by the presence of menaquinone in the culture medium which is the object of this application.

In the compositions used in the invention, the fermented media are used generally between 0.001% and 2% (p/p) by dry weight for the concentrated media, lyophilized or sprayed, generally between 0.1 and 10.0% (p/p) for the fermented media in liquid form.

The fermented medium can be used in any galenic form used in cosmetics or dermopharmacy: O/W and W/O emulsions, milks, lotions, pomades, capillary lotions, shampoos, soaps, sticks and pencils, sprays, body oils, without this list being limiting.

It is possible to incorporate the fermented medium into cosmetic vectors such as liposomes, chylomicrons, macro-, micro- and nanoparticles as well as macro-, micro- and nanocapsules, to absorb them on powdered organic polymers, talcs, bentonites and other mineral supports.

The fermented medium can be combined in cosmetic compositions with any other ingredient conventionally used in cosmetics: extraction and/or synthesis lipids, gelling and thickening polymers, tensioactive and emulsifying agents, hydro- or liposoluble active principles, extracts of other plants, tissue extracts, other marine extracts.

The cosmetic or dermopharmaceutical compositions containing the fermented medium can be creams, balms, or gels or lotions or sun creams and after-sun creams, aftershave products, depilatory or post-depilatory creams.

These cosmetic or dermopharmaceutical compositions containing the fermented medium are adapted for skin care, particularly to guard against the deleterious effects of free radicals and against all their consequences on cutaneous, physiological or premature aging, during exposure to natural or artificial UV as well as for care of the skin, and in particular the face, the body, the scalp and the hair; to modulate the cutaneous concentration of ceramides, to stimulate the immune system, to obtain a protective effect by the chaperone effect and as detoxifiers and anti-radicals with respect particularly to ozone.

These cosmetic or dermopharmaceutical compositions containing the fermented medium are used for the preparation of medications adapted for the care of the skin, particularly to guard against the deleterious effects of free radicals and against all their consequences on cutaneous, physiological or premature aging, during exposure to natural UV as well as for the care of the skin, in particular the face, the body, the scalp and the hair; to modulate the cutaneous concentration of ceramides, to stimulate the immune system, to obtain a protective effect by the chaperone effect and for detoxifying and anti-radical activity with respect particularly to ozone.

The fermented medium can also be used alone, or incorporated in cosmetic or dermopharmaceutical compositions, bound or incorporated or absorbed or adsorbed in the form of macro-, micro- and nanoparticles or in macro-, micro- and nanocapsules for application on or in textiles, synthetic or natural fibers, wool and any material adapted to be used to produce clothing or underclothing for daywear or nightwear, directly in contact with the skin or the hair to provide continuous topical application.

1. Process for the production of proteins by fermentation of micro-organisms, then extraction of the produced proteins, characterized in that the micro-organisms are of the family Thermus.

2. Process for the production of proteins according to claim 1, characterized in that the micro-organisms are from marine waters located adjacent hydrothermal sources of great depth.

3. Process for the production of proteins according to any one of claims 1 or 2, characterized in that the micro-organisms are of the strain GY1211, deposited under order number I-2715 in the National Culture Collection of Micro-organisms of the Pasteur Institute of Paris.

4. Process for the production of proteins according to any one of the preceding claims, characterized in that the fermentation is carried out in a nutritional medium having a pH of about 7.2 and including yeast extract at a concentration comprised between about 0.25% and 1%, ammonium chloride, sodium chloride at a concentration not exceeding 1%, and oligo-elements.

5. Process for the production of proteins according to any one of the preceding claims, characterized in that the fermentation takes place between about 72 and 75° C., with agitation.

6. Process for the production of proteins according to any one of the preceding claims, characterized in that the fermentation is followed by a detersive treatment permitting extraction of the proteins.

7. Process for the production of proteins according to any one of the preceding claims, characterized in that the fermentation is followed by a step of filtration at 0.2 μm and/or an ultrafiltration step.

8. Mixture of proteins obtained by the process according to one of the preceding claims, characterized in that the proteins are thermostable up to 100° C.

9. Mixture of proteins according to claim 8 characterized in that it includes menaquinone.

10. Mixture of proteins according to claim 8 characterized in that it has activities that are enzymatic peroxydasic, catalase-like, antiradical, inhibitive of glycation, and modulation of the synthesis of collagen.

11. Cosmetic or dermopharmaceutical composition characterized in that it includes a mixture of proteins according to one of claims 8 to 10.

12. Cosmetic or dermopharmaceutical composition according to claim 11 characterized in that the fermented media are used generally between 0.001% and 2% (p/p) by dry weight for concentrated, lyophilized or sprayed media, generally between 0.1 and 10.0% (p/p) for fermented media in liquid form.

13. Cosmetic or dermopharmaceutical composition according to one of claims 10 to 12, characterized in that it is present in any galenic form employed in cosmetology or dermopharmacy: O/W and W/O emulsion, milk, pomade, capillary lotion, shampoo, soap, stick and pencil, spray, body oil.

14. Cosmetic or dermopharmaceutical composition according to one of claims 10 to 13, characterized in that the mixture is incorporated in cosmetic vectors as liposomes, chylomicrons, macro-, micro- and nanoparticles as well as macro-, micro- and nanoparticles or absorbed on powdered organic polymers, talcs, bentonites or other mineral supports.

15. Cosmetic or dermopharmaceutical compositions according to one of claims 10 to 14, characterized in that the mixture is used with any other ingredient conventionally used in cosmetics: extracted and/or synthesized lipids, gelifying polymers and thickeners, surfactants and emulsifiers, hydro- or liposoluble active principles, extracts of other plants, tissue extracts, other marine extracts.

16. Cosmetic or dermopharmaceutical composition according to one of claims 10 to 15, characterized in that it is in the form of a cream, balm or gel, lotion or sun cream or after-sun cream, aftershave product, depilatory or post-depilatory cream.

17. Cosmetic or dermopharmaceutical composition according to claims 10 to 16, adapted for the care of the skin, particularly to guard against the deleterious effects of free radicals and against all their consequences on cutaneous, physiological or premature aging, during exposure to natural or artificial UV as well as for care of the skin, in particular the face, the body, the scalp and the hair, to modulate the cutaneous concentration of ceramides, to stimulate the immune system, to obtain a protective effect by the chaperone effect and as a detoxifier for the different hyper-reactive free radicals, particularly ozone.

18. Use of the mixture according to claims 8 to 10 or of a cosmetic or dermopharmaceutical composition according to claims 11 to 17, for the preparation of medications adapted for skin care, particularly to guard against the deleterious effects of free radicals and against all their consequences on cutaneous, physiological or premature aging, during exposure to natural UV or else for the care of the skin, in particular the face, the body, the scalp and the hair.

19. Use of the mixture according to claims 8 to 10 or of a cosmetic or dermopharmaceutical composition according to claims 11 to 17, bound or incorporated or absorbed or adsorbed in the form of macro-, micro- and nanoparticles or in macro-, micro- and nanocapsules, in textiles, synthetic or natural fibers, wool and any material adapted to be used to produce clothing or underclothing for nightwear or daywear, directly in contact with the skin or the hair to permit continuous topical application.
